(19) United States
(12) Patent Application Publication
(10) Pub. No.: US 2019/0083090 A1
(43) Pub. Date: Mar. 21, 2019

(54) SURGICAL STABILIZER

(71) Applicant: Quickring Medical Technologies, Ltd., Haifa (IL)

(72) Inventor: Simcha Milo, Haifa (IL)

(21) Appl. No.: 15/536,622

(22) PCT Filed: Dec. 17, 2015
(86) PCT No.: PCT/IB2015/002365

§ 371 Date: Jun. 15, 2017

Related U.S. Application Data
(60) Provisional application No. 62/093,755, filed on Dec. 18, 2014.

(51) Int. Cl. A61B 17/064; A61B 17/068

(52) U.S. Cl. CPC A61B 17/0644; A61B 2017/00566; A61B 17/0682

(57) ABSTRACT

A surgical tool for minimally invasive surgery is provided, where the tool includes a control handle; at least one vacuum arm deployable from a retracted position to an operative position for engagement with tissue in order to stabilize the tissue with respect to the tool; and a shaft movable with respect to the control handle, the shaft having an operating head movable from a retracted position to an operative position for performing a surgical function on the tissue stabilized by the at least one vacuum arm.

### CROSS-REFERENCE TO RELATED APPLICATION

This application claims the benefit of U.S. Provisional Application No. 62/093,755, filed Dec. 18, 2014, which is hereby incorporated by reference in its entirety.

### FIELD

Vacuum-assisted surgical stabilizers are described herein, and in particular, vacuum assisted surgical stabilizers suitable for use in minimally invasive surgery, such as repairing pathology of a heart valve within a cardiac chamber while the heart is still beating.

### BACKGROUND

Various devices and tools have been developed for carrying out percutaneous minimally invasive surgery within the human body. When an organ or tissue is moving, such as a beating heart, a surgeon can be required to time operation or manipulation of the tool in synch with movement of the organ. While certainly within the skill of many surgeons, an undesirable degree of variance can be introduced, which can be exacerbated for procedures that require a sequence of repetitive steps. Cardiac stabilizers or immobilizers are medical devices used to hold still or immobilize an area or part of the heart while it is still beating, so that precise surgical procedures (such as anastomosis during bypass surgery) can be performed on it while it is still moving.

### SUMMARY

A surgical tool is provided that is configured to be fixed relative to an organ or tissue in order to stabilize the tool relative to the tissue, which is particularly advantageous if the tissue is moving, as is the case of a beating heart, for example. This stabilization can be such that the tool moves with the tissue or, more preferably, such that the tool can prevent or at least reduce movement of a localized zone of the tissue to which the tool is fixed. The latter can be accomplished by also fixing the tool to a structure in the operating room, such as an operating table.

The tool also includes a manipulative component that can be moved to perform surgical functions on the stabilized tissue when the tool is stabilizing the tissue. The operating end of the tool, which can include both the manipulative component as well as structure for stabilizing the tissue, are configured for minimally invasive surgery and, as such, can be inserted each in a compact configuration into a patient, such as using a guidewire, and then the structure for stabilizing the tissue deployed to stabilize the tissue, followed by use of the manipulative component. Once the procedure is complete, the structure for stabilizing the tissue and the manipulative component can be moved back into the compact configuration or a different compact configuration and withdrawn from the patient.

The tool, in the exemplary form of an intra-cardiac tissue stabilizing device, has at least two different modes. In the first, retracted, mode, the tool has a narrow, elongated shape, to enable insertion and operation in a beating heart. In the second, operating, mode, the tool has a physically different shape that is altered by deployment of at least one stabilization arm. The at least one stabilization arm can be attached to tissue, such as by using suction, to thereby fixate the tool. When stabilization is over, the device has to again change shape to the first, retracted, mode in order to be removed from inside the beating heart. In the first or retracted mode the device has to have a very narrow profile, so that it can be introduced into the heart through a very small hole, or through a blood vessel reaching the heart. It can also have an externally smooth surface, so as to reduce or eliminate snagging on cardiac tissue or causing tears in the tissue while the device is advanced to arrive at the area inside the heart intended for stabilization.

Preferably, though not necessarily, while in operating mode, the device should be able to connect to an externally anchored platform, such as the operating table, the floor of the room etc. so as to provide stabilization to the desired area inside the heart relative to the platform to which it is anchored.

Also desirable is to be able to use the stabilizer to orient other tools used in the same procedure inside the heart. The stabilizer remains in a fixed position relative to the target area of the tissue, so any device which remains fixed relative to the stabilizer is also fixed relative to the tissue, and any movement relative to the stabilizer is an identical movement relative to the tissue. This feature may help guide other device which are meant to perform a procedure on cardiac tissue such as suturing, staple insertion, ablation (e.g., cold, hot, RF), injection (e.g., of stem cells, drugs, biological clue, biological scaffold material), delivery of a construct (such as artificial valve, tissue anchor, artificial chordae) to accurately target the position they need in the heart despite the blood obscuring vision and the constant movement of the heart.

### BRIEF DESCRIPTION OF THE DRAWINGS

FIG. 1 is a perspective view showing an alternative embodiment of a surgical stapler similar to that of the prior embodiment, but additional configured for stabilizing the area to which the staples are deployed, showing the introducer in a deployed configuration;

FIG. 2 is a perspective view of the alternative embodiment of the surgical stapler of FIG. 1, showing the operation of a knob for controlling rotation of an elongated barrel;

FIG. 3 is a perspective view of the alternative embodiment of the surgical stapler of FIG. 1, showing the stapler attached to a flexible arm for stabilizing the surgical stapler;

FIG. 4 is a perspective view of the underside of the surgical stapler of FIG. 1;

FIG. 5 is an enlarged fragmentary view of the introducer of the surgical stapler of FIG. 1 in a partially retracted position;

FIG. 6 is an enlarged fragmentary view of the introducer of the surgical stapler of FIG. 1 in a further retracted position;

FIG. 7 is an enlarged fragmentary view of the introducer of the surgical stapler of FIG. 1 in a fully retracted position to expose suction arms and optional remote suction pods;

FIG. 8 is an enlarged fragmentary view of the end portion of the surgical stapler of FIG. 1 showing an exposed stapling head and the partial deployment of the suction arms and the suction pods in a retracted position;

FIG. 9 is an enlarged fragmentary view of the end portion of the surgical stapler of FIG. 1 showing the exposed stapling head and the full deployment of the suction arms and partial deployment of the suction pods;

FIG. 10 is an enlarged fragmentary view of the end portion of the surgical stapler of FIG. 1 showing the exposed stapling head and the full deployment of the suction arms and full deployment of the suction pods;

FIG. 11 is an enlarged fragmentary view of the end portion of the surgical stapler of FIG. 1 showing the exposed stapling head and the full deployment of the suction arms and full deployment of the suction pods, similar to what is shown in FIG. 10 but from a different angle;

FIG. 12 is an enlarged fragmentary view of the end portion of the surgical stapler of FIG. 1 showing an underside of the suction arms;

FIG. 13 is a perspective view of the surgical stapler of FIG. 1 inserted into a mitral valve and showing the vacuum arms in an almost retracted position;

FIG. 14 is a perspective view of the surgical stapler of FIG. 1 inserted into a mitral valve and showing the vacuum arms in a more deployed position as compared to FIG. 13;

FIG. 15 is a perspective view of the surgical stapler of FIG. 1 inserted into a mitral valve and showing the vacuum arms in a more deployed position as compared to FIG. 14 and the stapler head pivoted away from the stapler body;

FIG. 16 is a perspective view of the surgical stapler of FIG. 1 inserted into a mitral valve and showing the vacuum arms fully deployed and engaged with the annulus of the valve and the stapler head pivoted away from the stapler body and positioned to implant a staple;

FIG. 17 is a perspective view of the surgical stapler of FIG. 1 inserted into a mitral valve and showing the vacuum arms fully deployed and engaged with the annulus of the valve, a first staple having two rings having been implanted, and the stapler head pivoted toward the stapler body and positioned to receive another staple to implant; and

FIG. 18 is a perspective view of the surgical stapler of FIG. 1 inserted into a mitral valve and showing the vacuum arms fully deployed and engaged with the annulus of the valve and the stapler head pivoted away from the stapler body and positioned to implant a staple adjoining the already-implanted staple in order to form a chain of staples.

### DETAILED DESCRIPTION

A surgical tool, in the exemplary form a surgical stapler, is provided that is provides for fixing of the stapler relative to the tissue adjacent to where a staple is to be implanted. The stapler is configured to be used while a heart is beating. However, the beating of the heart means that the target location for implanting the staples will also be moving. In order to facilitate implantation of the staples, particularly when the leg of one staple must be inserted through the ring attached to an already-implanted staple, the alternative surgical stapler can advantageously be fixed relative to the tissue. This is accomplished at least in part by using one or more deployable vacuum arms 107 and suction pods 108, as discussed in detail herein. Optionally, to provide further stabilization, the stapler can be modified to also be fixed to an external object, such as an operating table. Thus, not only can relative movement between the stapler and the tissue be reduced, but also movement of the stapler and tissue can be reduced to facilitate use of the stapler by a surgeon.

While discussed in the context of a surgical stapler, the vacuum arms 107 and/or suction pods 108 can together or separately be incorporated into other forms of staples and, indeed, other surgical tools. Broadly, a tool—such as the illustrated surgical stapler 111—can be provided that includes a control handle, at least one vacuum arm deployable from a retracted position to an operative position for engagement with tissue in order to stabilize the tissue with respect to the tool; and a shaft movable with respect to the control handle, where the shaft has an operating head movable from a retracted position to an operative position for performing a surgical function on the tissue stabilized by the at least one vacuum arm. As will be appreciated, such structures can be incorporated into a variety of surgical tools for minimally invasive surgical procedures in addition the stapler described herein.

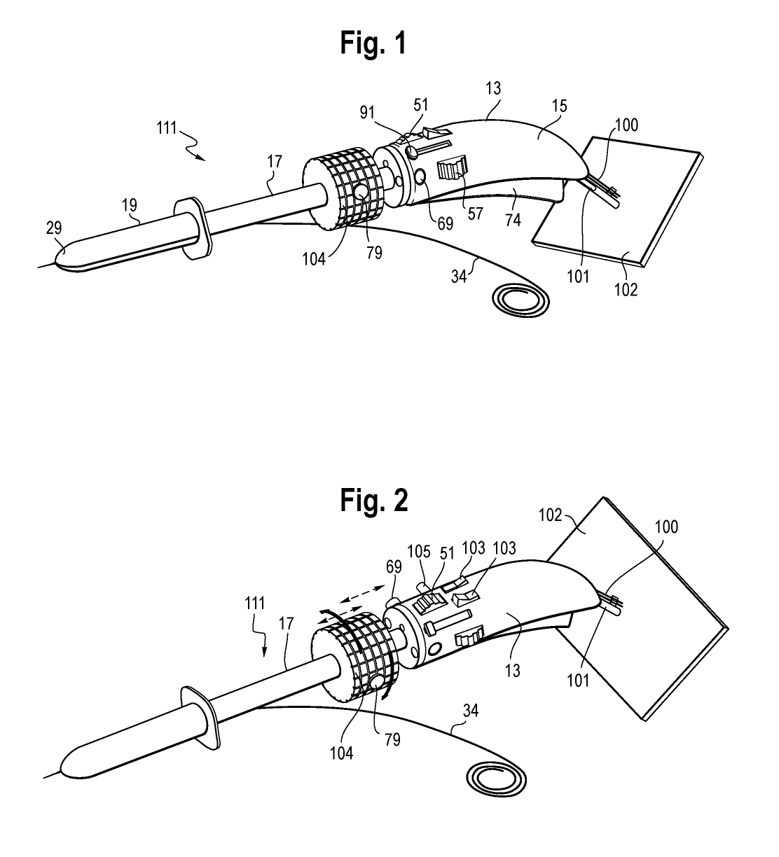

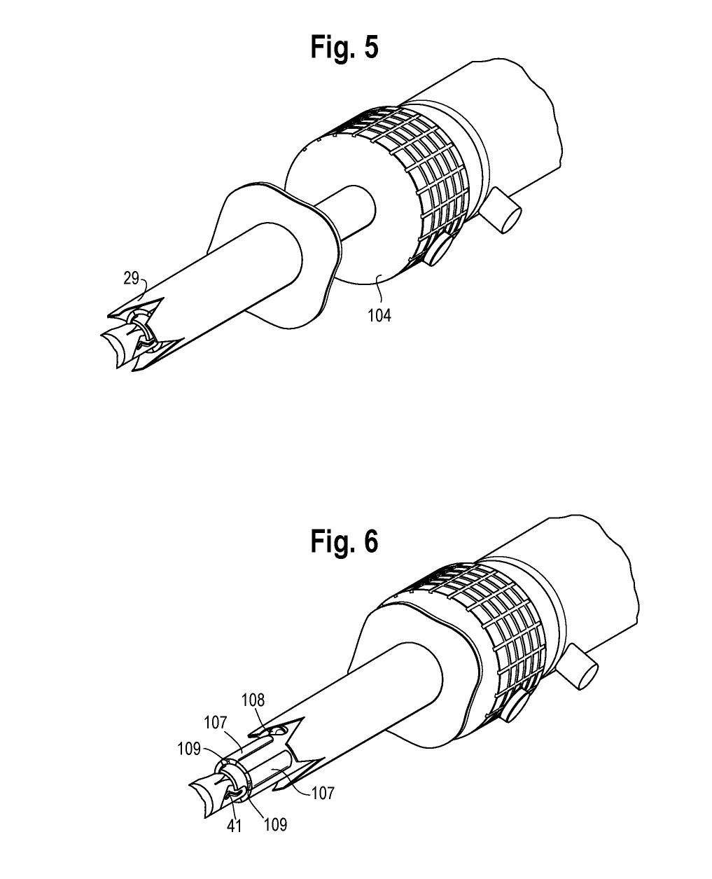

Turning to details of the stapler 111, and with reference to FIGS. 1-12, the surgical stapler 111 is configured to be fixed relative to tissue adjacent the stapler implant site and, optionally, relative to a stable external object, such as an operating table. In many ways the alternative surgical stapler 111 is similar to the surgical stapler 11 discussed in WO 2015/063609, published May 7, 2015, which is hereby incorporated by reference in its entirety (“the '609 publication”). The structure and operation of the surgical stapler 111 is similar to that described in the '609 publication, with like reference numbers indicating like components, with differences relating to the stabilization and immobilization function discussed in detail herein.

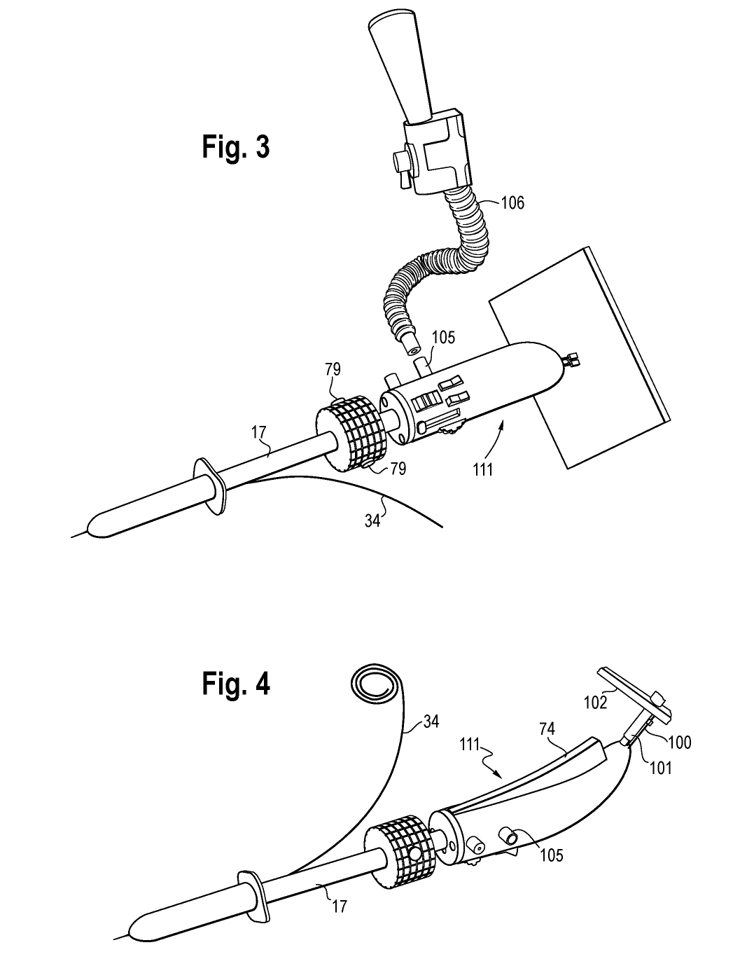

The control handle 13 of the alternative stapler 111 can include a connecting site 105 for connection of a mounting arm 106, as illustrated in FIG. 3. The connecting site 105 and mounting arm 106 can be of any suitable structure, and function to fix the control handle 13 and thus the alternative stapler 111 relative to an external object, such as an operating table. The mounting arm 106 can be flexible, rigid, or a combination, e.g., moveable components that can be locked against further movement once in a preferred position. One example of a suitable arm is disclosed in U.S. Pat. No. 3,858,578.

The alternative stapler 111 includes a barrel 17 with a stapling device 21 at a distal end thereof. However, instead of using the knurled wheel 72 to rotate the barrel 17, a barrel knob 104—spaced from the grip 15—is used to rotate the barrel 17 and thus the stapling device 21, as shown in FIG. 1. Also, instead of having the triggers 79 disposed substantially coaxially on opposite sides of the grip portion 15 of the handle 13, as in the prior embodiment, the triggers 79 of the alternative embodiment of the stapler 111 are disposed on the barrel knob 104, as shown in FIGS. 1-3. As with the prior embodiment, simultaneous pressing of the two oppositely disposed triggers 79 is required to actuate the trigger mechanism 77. This spatial disposition of a pair of coaxial triggers 79 is selected so that a surgeon, using a thumb and index finger for rotation of the knob 104, need only slightly move their thumb and finger to depress the triggers 79. As described with respect the prior embodiment, the trigger mechanism 77 actuates the spring-loaded former mechanism 71 which presses the upper surfaces of the shoulders of the M-shaped staple 47 forward, causing the U-shaped crown connector with its undersurface resting on the anvil 75 to be reshaped into a straight connector.

When the control handle 13 is fixed, then the control handle 13 cannot be used by a surgeon to axially move the barrel 17. To allow for the barrel 17, and thus the stapling device 21, the barrel 17 is disposed within an outer tube or sleeve 109, as shown in FIGS. 35-38. The barrel 17 is both axially slidable and rotatable within the outer tube 109 upon manipulation using the barrel knob 104, as indicated in FIG. 2. A proximate end of the outer tube 109 is attached to the control handle 13, and an open, distal end of the outer tube 109 is positioned adjacent the stapling device 21 at the distal end of the barrel 17. Optionally, the rotation of the barrel 17 within the outer tube 109 can be limited, for example, to about 90 degrees. Also optionally, the axial movement of the barrel 17 relative to the outer tube 109 can be limited, for example, to about 10-12 mm.

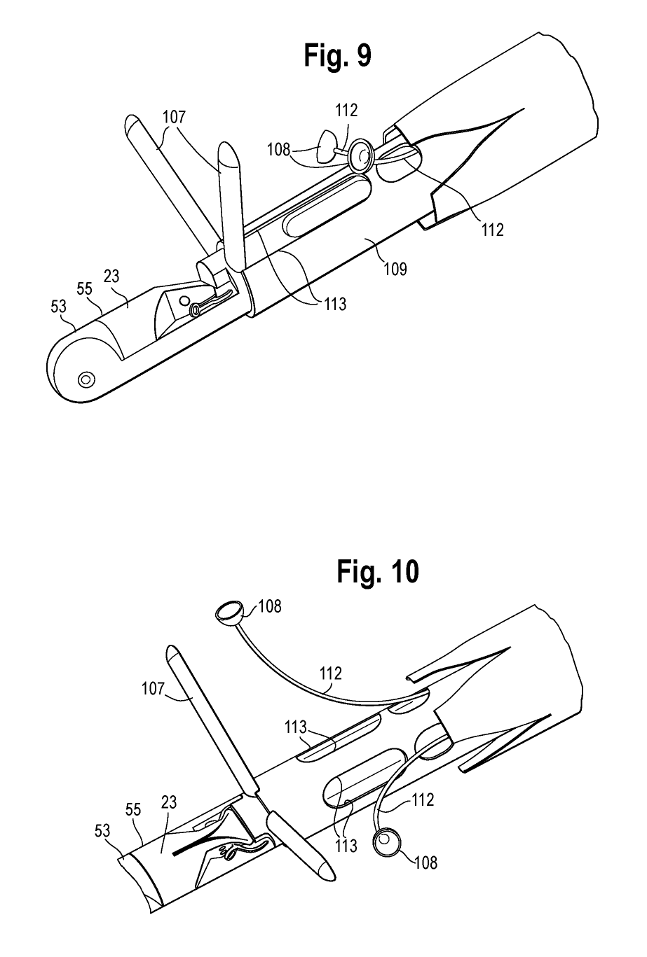

Pivotably connected to the distal end of the outer tube 109 are a pair of vacuum arms 107. The vacuum arms 107 are movable from a retracted position, illustrated in FIG. 6, to a fully deployed position, illustrated in FIGS. 9-12. Once deployed, vacuum can be applied to the vacuum arms 107 to secure the arms 107, and thus the outer tube 109 and control handle 13, to tissue adjacent the stapling site. So deployed and with vacuum applied, the stapler 111 can move with the beating of the heart. However, this can be counteracted to some extent by manipulation of the stapler 111 by a surgeon and, more preferably, by fixing the stapler 111 using the arm 106, as discussed above. With the stapler 111 fixed relative to an external object, and, in particular, the control handle 13 and outer tube 109 fixed relative to an external object, and with the vacuum arms 107 deployed and fixed by vacuum to the tissue adjacent to the staple implant site, the beating of the heart can cause little to no movement at the staple implant site. This advantageously allows for a surgeon to use the stapler 111, and in particular to axially and rotationally manipulate the barrel 17 and, as discussed above with respect to the prior embodiment, implant one or more staples. Such stabilization of the staple implant site can be particularly beneficial as a surgeon is guiding the tip of a leg of a staple into the O-ring of an already-implanted staple.

The vacuum arms 107 define a triangular area between them when deployed and engaged with the tissue adjacent the implant site. The stapler 111 can be configured so that one and preferable several staples can be implanted with the vacuum arms 107 engaged with the tissue adjacent a given implant site, such as by using the barrel knob 104 to axially and rotationally manipulate the barrel 17 to position the stapling device 21 to sequentially implant staples, such as the exemplary three staples illustrated in FIG. 12. Once a different triangular tissue area is desired for further implanting of staples, the vacuum arms 107 can be disengaged from the tissue by discontinuing the application of vacuum, and the stapler 111 manipulated to rotate the vacuum arms 107 to engage tissue adjacent the next implant site.

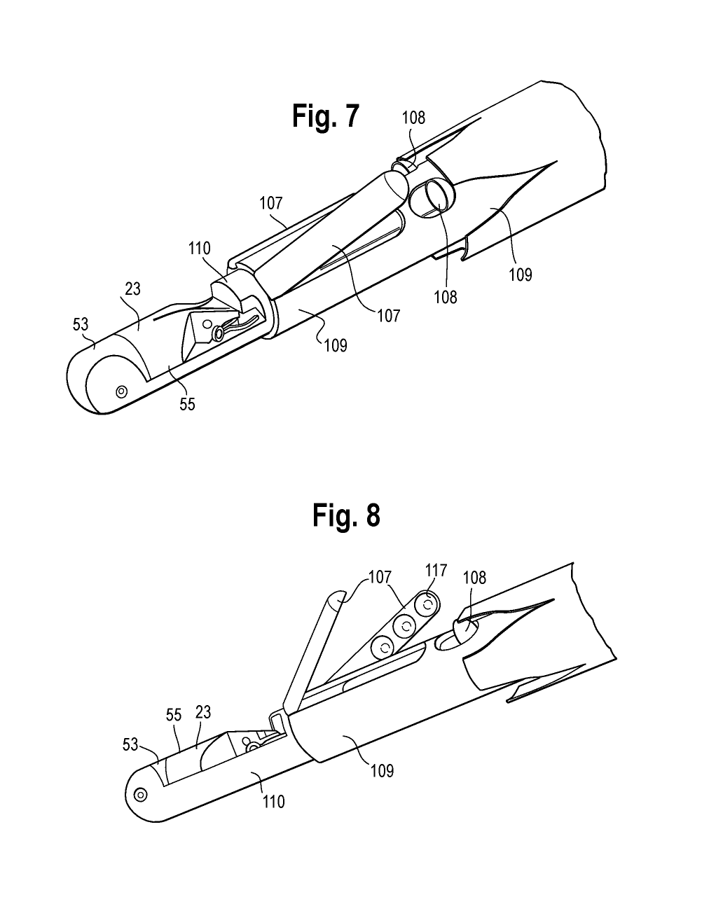

Each of the vacuum arms 107 has an end pivotably connected, such as by a hinge pin, relative to the outer tube 109, as depicted in FIG. 7. The vacuum arms 107 preferably have a thickness such that they do not protrude much, if at all, beyond the outside diameter of the outer tube 109, as shown in FIG. 6. Indeed, the vacuum arms 107 can each be recessed within an associated slot 113 of the outer tube, as evident by comparing FIGS. 6-9. Furthermore, grooves can be provided in the barrel 17 to provide space to accommodate the vacuum arms 107 in their closed position in order to lower their external contour and minimize the diameter of the stapler 111. The underside of each of the vacuum arms 107, which will engage with the tissue, is flat but for a series of spherical dimples 115, as shown in FIGS. 8 and 12. Those dimples 115 are interconnected at their bottoms by a vacuum passage 114, as shown in FIG. 12. The vacuum passage 114 is in turn operatively connected to a vacuum source 102, such as through vacuum tubing 101, as shown in FIG. 1, running through the interior of the barrel 17. The vacuum supply reaches the arms 107 by pliable tubes 119, shown in FIGS. 6 and 7, that extend through openings in the barrel 17 and into the interior of the barrel 17, where they are routed in operative connection, whether direct or indirect, to the vacuum source 102. A fluid trap can be provided between the stapler 111 and the vacuum source 102. When vacuum is applied, tissue can be partially drawn into the dimples 115, thereby securing the arms 107 and thus the outer tube 109 and the control handle 13 relative to the tissue. Each of the vacuum arms 107 can have an associated actuator 103 on the control handle 13, as shown in FIG. 2, for deploying and retracting the arms 107. As shown in FIG. 2, one of the actuators 103 is a closed position, depressed toward the rear of the control handle 13, which corresponds to a retracted or closed position of one of the arms 107. The other of the actuators 103 is depressed toward the barrel 17, which, if the introducer were retracted, would correspond to an open or extended position of one of the arms 107. The actuators 103, and thus the arms 107, are not limited to fully retracted and fully extended positions; rather, they can be adjusted therebetween. This advantageously can allow the surgeon to control the angle, the direction and the rate of the vacuum arm movements.

While the vacuum arms 107 are configured to engage the tissue adjacent the implant site from one side, it can be advantageous to provide optional additional stabilization support from an opposite side, the ventricular side. To this end, a plurality of remote suction pods 108 are provided on the stapler 111. The remote suction pods 108 can be deployed from the stapler 111 via tubular elements that both supply vacuum as well as guide the deployment of the pods 108, as will be discussed further herein. More specifically, the pods 108 can provide for stabilization from the (left ventrical) ventricular side.

It will be understood that the remote suction pods 108 are not limited to use in conjunction with the vacuum arms 107, nor are the remote suction pods 108 limited to use with the stapler 111 for the purposes described herein. Instead, the remote suction pods 108 can be utilized on any suitable minimally invasive device where stabilization is desired.

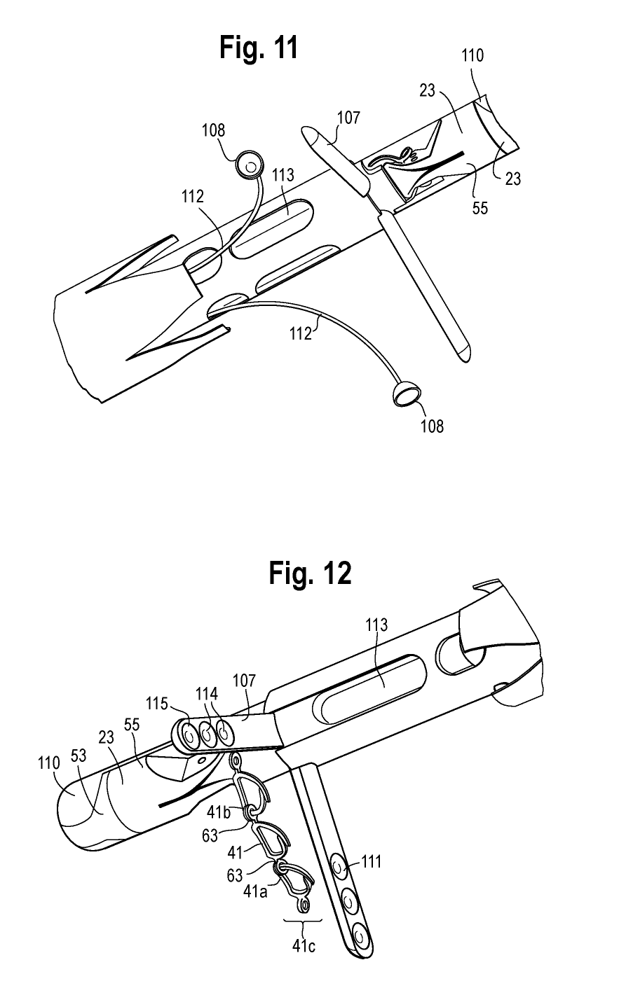

The remote suction pods 108, according to an exemplary embodiment, can be partial spherical cup-shaped in appearance, as shown in FIGS. 38-40, with a vacuum tube 112 operatively connected for providing vacuum. The vacuum tube 112 can in turn be operatively connected to the vacuum source 102. The vacuum tubes 112 are preferably not just a flexible tube, however, but rather have a sufficient degree of rigidity such that can push the pods 108 from a retracted position, illustrated in. FIGS. 7 and 8, to a deployed position, illustrated in FIG. 11. In a preferred, but non-limiting example, the vacuum tubes 112 can be formed of a material having a shape memory, such as may be thermally imparted, so as to provide the desired curve and thus approximate position of the pods 108 when deployed. For example, the vacuum tubes 112 can be formed of Nitinol (Nickel Titanium memory alloy) tubes thermally imparted to have the desired curve when fully deployed.

The suction pods 108 can be manipulated and activated from the space outside the stapler 111, between the tip of the handle and the polymeric plate 102. In particular, each of the vacuum tubes 112 has an extension 100 (whether integral or in communication therewith), that can be fed into or pulled out of the control handle 13 in order to extend and retract, respectively, the section pod 108 on the end thereof. Manipulation of the extension 100 and deployment and retraction of the vacuum tubes 112 can be similar in feel to a surgeon as the use of a guidewire. For example, the surgeon, holding the relevant extension 100 between his thumb and index fingers, can push the relevant extension 100 forwards and further into the control handle 12, which will, via the vacuum tube 112, dislodge the suction pod 108 from its seat for deployment as desired, including rotation. Once the location is satisfactory, vacuum can be applied to the pod 108 also from the same area where he holds the Nitinol tube or extension.

The direct and independent control of the suction pods 108, via their associated vacuum tube 112 and extension 100 extending outwardly from the rear of the control handle 13, beneficially allows for precise application and adjustment by a surgeon. For instance, to increase stabilization or immobilization, the surgeon might wish to apply tension on the pods 108 that are vacuum engaged to the tissue. This can easily be accomplished by slightly pulling backwards on the extensions 100 of the vacuum tubes 112. Once positioned, the extensions 100 and thus the vacuum tubes 112 can be fixed to the handle for the time of the procedure. Retraction of the pods 108 back into their seat, shown in FIG. 6, happens similarly to deployment, however in reversed motions.

The surgical stapler 111 can be adapted for a variety of endoscopic uses, including for effecting annuloplasty of a heart valve, particularly the mitral valve, as described in the '609 publication. The stapler 111 can be used for application to a beating heart through its apex into the left ventricle. Once a guide wire 34 used for guiding insertion is withdrawn, the elongated barrel 17 of the stapler is moved relative to the sheath 19 so that the stapling device 21 begins to emerge from the distal end of the opened split tip 29 of the sheath 19.

The stapling head 23 is pivoted away from its at rest juxtaposition with the stapler body 110, and the end portion is rotated so as to position the staple 41, carrying two rings 63, so it is aligned with the mitral valve annulus at about the midpoint of the posterior leaflet. This very first staple 41 is equipped with bilateral rings as seen in FIG. 12, the middle staple in the interlocked chain, is implanted in this location, and the stapling head 23 is then reloaded with one staple 41a and 41b at a time from the magazine deploying each next staple on alternating sides, to create thus an optionally symmetrical constricting chain 41c of interconnected staples with the number desired by the surgeon along the valve annulus to achieve the desired amount of constriction of the annulus, as described generally in U.S. Pat. No. 8,123,801 and in the '491 patent. The objective of the procedure is to counteract the pathological condition of the mitral valve in order that the leaflets again co-apt to effectively close the valve and prevent, or at least minimize, regurgitation during the pumping stroke, Systole, of the left ventricle.

The pivoting movement of the stapling head 23 is effected by the control handle by rotation of a knurled wheel 51 on the grip portion 15 of the control handle 13. This knurled wheel 51 at the upper ridge of the grip portion 15 of the control handle connects via mechanism that traverses the length of the elongated barrel 17 to cause the pivoting of the stapling head 23.

The surgical stapler 111 is optionally equipped with a crimping mechanism which can effectively change the spacing between the sharpened prongs at the end of the two legs of a staple. The mechanism is located in association with the holder region in the end section of the stapling head 23, and it is designed to apply inward lateral pressure to the exterior lateral surfaces of the stiff legs of the staple 41 to deform them toward each other. For example, the staples may be made from stainless steel or from Co—Cr alloy of comparable stiffness. For instance, the staples 41 initially loaded in the surgical stapler might be formed so the sharpened tips are spaced from each other about 7.5 mm, and the crimping mechanism can reduce spacing, for example to about 5.5 mm, which might be about the length of the crown connector in its straightened implanted form. The crimping mechanism is operated by a slide 91 located on the grip portion 15 of the handle 13. Movement of the slide 91 proximally from its at rest position effects laterally inward bending of the legs of the staple then loaded in the stapling head 23 via linkage that extends through the elongated barrel 17. The stapling head 23 is formed with a base section 53 and a rotatable end section 55. The rotation of the end section 55, which carries the staple 41, relative to the base section 53 is controlled by another knurled wheel 57 located on the left hand side of the grip portion 15 of the control handle 13, which likewise contains linkage extending through the barrel 17. The delivery of the staple 41 is actuated by the lever 74 of the handle 13.

The hollow barrel 17 includes a plurality of staples disposed in a cylindrical holder or magazine and aligned so that each staple lies with its body in a common plane that preferably includes the central axis of the barrel. The staples in the magazine have only a single ring disposed laterally from one leg. For convenience of the surgeon, the stapler 11 is designed so that the magazine can be rotated 180° so that the ring-carrying leg of the staple is at the right hand edge of the staple in the barrel of the stapler. Rotation of the magazine for 180° is effected by a slide 69 near the distal end of the grip portion 15 of the control handle 13. The slide 69 can be moved transversely across the diameter of the grip portion 15 and is arranged so that when the end of the slide 69 protrudes from the left hand side of the grip portion, the staples are orientated with the ring-carrying leg to the left. When the slide 69 is pressed inward to the right so that it protrudes from the right hand surface of the grip portion 15, the magazine has been rotated 180° so that the ring is now attached to the leg on the right. This can result in omitting the need of rotating the entire, fixed delivery device in 180° in order to enable symmetrical deployment on alternative sides of the initial, double O-ring 63, first staple 41.

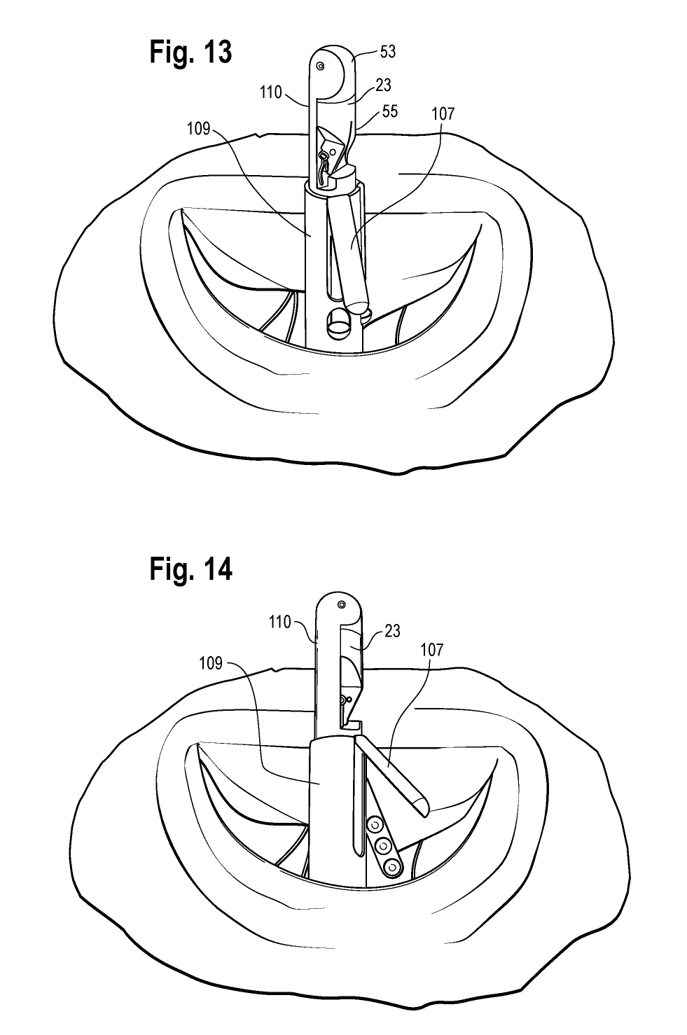

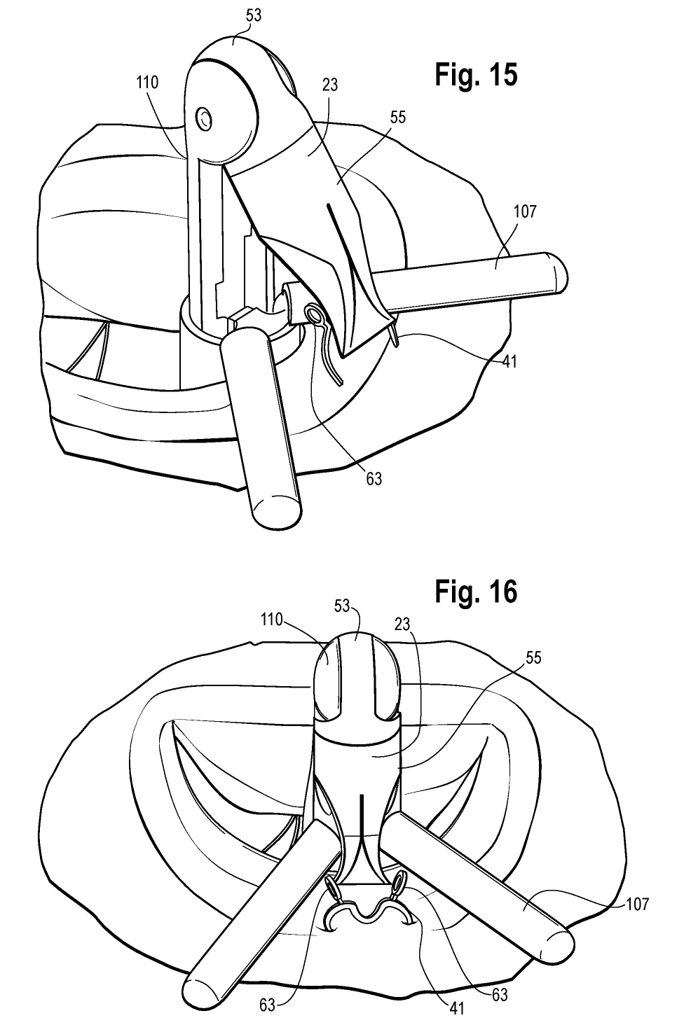

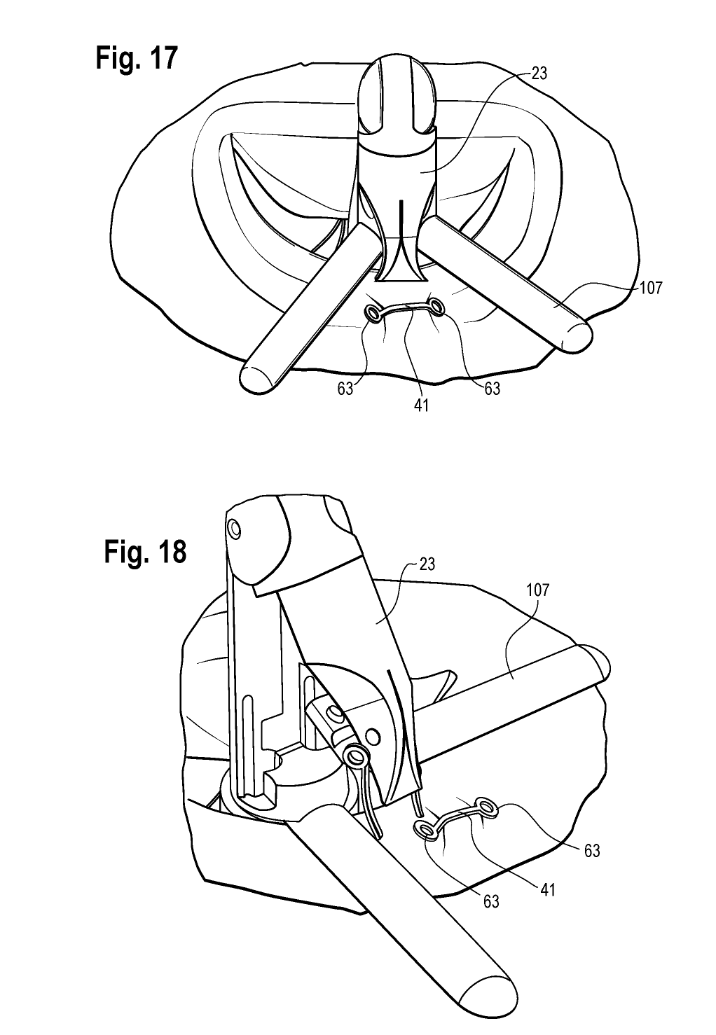

In use, the surgical stapler 111 is first inserted into a mitral valve with the vacuum arms 107 in a retracted position, as generally shown in FIG. 13. Next, the vacuum arms 107 are moved toward a deployed position, as shown in FIG. 14. The stapler head 23 can move toward a more deployed position, as shown in FIG. 15, by pivoting away from the stapler body 110. Before implanting a staple, the vacuum arms 107 can be engaged with the annulus via vacuum application to provide for stabilization, and, as shown in FIG. 16, the stapler head 23 pivoted away from the stapler body 110 and positioned to implant a staple 41. After the first staple 41 has been implanted, the stapling head 23 can be pivoted back toward the stapler body 110 to receive another staple 41a, as shown in FIG. 17, then the body 110 rotated and the stapling head 23 pivoted outwardly to position the another staple 41a for implantation, as shown in FIG. 18.

Although the invention has been described and illustrated in terms of the best mode presently understood by the inventors to perform such an annuloplasty, it should be understood that various changes and modifications to the devices illustrated made be made without departing from the scope of the invention, which is defined in the claims appended hereto. Furthermore, various features of the invention are emphasized in the claims that follow.

1. A surgical tool for minimally invasive surgery, the tool comprising:
a control handle;
at least one vacuum arm deployable from a retracted position to an operative position for engagement with tissue in order to stabilize the tissue with respect to the tool; and
a shaft movable with respect to the control handle, the shaft having an operating head movable from a retracted position to an operative position for performing a surgical function on the tissue stabilized by the at least one vacuum arm.

2. The surgical tool of claim 1, comprising at least two vacuum arms and wherein the operating head is configured to be movable to perform the surgical function on the tissue stabilized between the two vacuum arms.

3. The surgical tool of claim 1, wherein the retracted position of the at least one vacuum arm and the retracted positions of the operating head have a reduced cross section as compared to the operative positions thereof such that, in the retracted positions, they can be inserted into and removed from a patient.

4. The surgical tool of claim 1, wherein the shaft is rotatable and axially extensible with respect to the control handle and the at least one vacuum arm.

5. The surgical tool of claim 4, wherein the operating head is pivotable and rotatable with respect to the shaft.

6. The surgical tool of claim 5, wherein the pivoting of the deployment of the at least one vacuum arm, the rotation of the shaft, the extension of the shaft, the pivoting of the operating head, and the rotation of the operating head are each independently controlled using the control handle.

7. The surgical tool of claim 6, wherein the rotation of the shaft is actuated using a knob of the control handle.

8. The surgical tool of claim 7, wherein the extension of the shaft is actuated using the knob.

9. The surgical tool of claim 8, wherein the operating head includes a movable element that can be manipulated using one or more actuators disposed on the knob.

10. The surgical tool of claim 9, wherein the tool is a surgical stapler, and the movable element is configured to implant a staple.

11. The surgical tool of claim 1, further comprising at least one remote suction pod moveable from a retracted position to a deployed position engageble with tissue upon application of a vacuum.

12. The surgical tool of claim 11, wherein each of the at least one remote suction pod is mounted on an extendible flexible tube that is connectable to a vacuum source.

13. The surgical tool of claim 12, wherein extension of the flexible tube can be used to control the position of the suction pod and tension tissue to which the pod is attached.

14. The surgical tool of claim 13, wherein the flexible tube is formed of a nickel titanium memory allow tube.

15. A method of repairing a patients leaking mitral valve using the surgical tool of any of the foregoing claims, which method comprises the steps of:
inserting the at least one vacuum arm and the operating head, in their retracted positions, into the left ventricle of a patient's heart through a transapical passageway and through the valve opening between the leaflets of the mitral valve into the left atrium; deploying the vacuum arms and engaging them using vacuum with tissue of the valve annulus adjacent to the mid-posterior leaflet to stabilize the tissue relative to the tool;
deploying the operating head and using the operating head to implant a first staple into the valve annulus adjacent the mid-posterior leaflet, whereby the tissue is constricted where the staple is implanted;
repeating the step of using the operating head to implant subsequent staples each connected to a previously implanted staple, whether the first stable or one of the subsequent staples, to create a chain that changes the shape of the mitral valve annulus so as to effect improved closing of the mitral valve;
ceasing the application of vacuum and moving the at least one vacuum arm to the retracted position and moving the operating head to the retracted position; and
withdrawing the at least one vacuum arm and operating head from the heart of the patient.

16. The method of claim 15, further comprising alternating sides of the first staple when implanting subsequent staples on order to create a symmetrical chain.

17. The method of claim 16, where in the first staple has bilateral rings, and wherein subsequent staples each have a ring, and wherein the staples are connected via the rings.
